(19) United States
(12) Patent Application Publication
(10) Pub. No.: US 2019/0314325 A1
(43) Pub. Date: Oct. 17, 2019

(54) Process and Method to Accelerate Cellular Regeneration, Healing and Wound Management

(71) Applicant: Richard Postrel, Miami Beach, FL (US)

(72) Inventor: Richard Postrel, Miami Beach, FL (US)

(21) Appl. No.: 16/234,799

(22) Filed: Dec. 28, 2018

Related U.S. Application Data
(63) Continuation-in-part of application No. 15/952,237, filed on Apr. 13, 2018.

(51) Int. Cl. A61K 31/352; A61P 17/02; A61K 36/185; A61P 29/00; A61K 9/00; A61K 9/70; A61P 21/02; A61K 31/137

(52) U.S. Cl. CPC A61K 31/352; A61P 17/02; A61K 36/185; A61K 31/137; A61K 9/0014; A61K 9/7015; A61P 21/02; A61P 29/00

(57) ABSTRACT

This invention provides compositions and methods for cellular regeneration and more rapid healing of surface lesions such as cuts, abrasions, rashes and other skin disturbances. A preferred consumer-based and battlefield embodiment features a spray-on composition delivering at least one substance that stimulates intercellular communication using tunneling nanotubes (TNTs). The induced TNTs increase transport of chemical and/or electrochemical information between more stable and nascent cells or between more intact and damaged cells. The inventive substance is applicable for use for ordinary household type injuries as well as industrial and/or battlefield events, perhaps from physical or chemical accidents or even several types of chemical weapons. Other preferred embodiments incorporate a sealant composition or coating to shield the affected surface area designated for healing from contamination and/or solvents/carriers to facilitate transdermal delivery of one or more pharmaceutically active ingredients.

This application is a continuation in part of application Ser. No. 15/952,237 the contents of which are incorporated in their entireties by reference.

This invention relates to formulations and methods for the use and application of treatments for surface wounds, lesions and/or injuries. Preferred embodiments include an easy to apply cannabinoid-based spray that accelerates nanotube tunneling extensions from healthy developed cells that reach out to assist damaged or nascent cells. Various preferred embodiments include a multi-functional compound spray with a cannabinoid-based molecule associated with an antibiotic, an analgesic, an antiseptic, an anesthetic, an ultra-violet absorbent, a coolant, an emollient and/or a colorant in a coating that dries to seal and protect the wounded surface. A formulation designed to facilitate transdermal delivery of one or more active ingredients of the composition may be preferred in some applications.

This invention provides tools to accelerate the healing of skin wounds, lesions and/or injuries. A preferred embodiment includes a portable spray applicator that delivers natural substances that stimulate skin cells to heal. For first responder or battlefield use several embodiments may be incorporated in a small kit. For irritants, including skin allergies, chemical exposure irritation, rashes, minor burns, etc. a spray on glaze-like overlay delivers the medicament and provides a protective coating while the underlying skin heals. For deeper or more extensive wounds a patch may be used for stronger binding and protection. For general consumer use sprays and patches may be separately obtainable. Additional preferred embodiments include a multi-functional compound spray wherein the cannabinoid-based molecule that stimulates the healing process is combined with one or more other ingredients such as: an antibiotic, an analgesic, an antiseptic, an anesthetic, an ultra-violet absorbent, a coolant, an emollient and/or a colorant in a coating that dries to seal and protect the wounded surface.

This invention may include embodiments that feature one or more composition effective in delivering pharmaceutically active agents through the skin including transdermally. Embodiments may include compositions formulated with multiple components that act additionally or synergistically to promote healing. Such components may include but are not limited to: non-steroidal anti-inflammatory agents—such as aspirin, ibuprofen, naproxin or ketoprofen; a muscle relaxant—such as cyclobenzaprine; an antimicrobial—such as anti-bacterial or antifungal, an enzyme activator or inhibitor which may have effects such as pain relief, improved healing rate, reduced scarring, improved control of circulation, pain control; cannabinolic compounds -such as endogenous and exogenous cannabinoids, their precursors; etc. Such formulations featuring rapid absorption into and through the skin may provide local relief from pain, muscle spasms, etc., in promoting healing processes.

### BACKGROUND OF THE INVENTION

When a person suffers an injury, has a skin reaction resulting in a surface lesion, such as a rash, chemical burn, abrasion, etc., or has surgery resulting in a surface wound, it is important that the wound be closed, patched, covered and/or protected so that it heals as quickly as possible. Rapid wound healing will, among other things, increase patient comfort, decrease the risk of the wound re-opening, decrease the risk of re-injuring the wound site and allow the patient to return to normal activities.

Skin and parts of our mucous membranes protect the body from the environment and maintain body temperature and moisture. Damaged skin may provoke a pain or itch sensation, but may also allow infectious organisms, harmful chemicals, and foreign particles to invade our bodies. Rapid healing to restore the protective properties of the skin and to eliminate the irritant sensations is thus essential for optimal performance. In the healing process the body sets in motion biochemical and biomolecular processes to restore damaged cells and generate new cells. After the injury, given time and protection, most wounds will heal.

Some wounds are especially difficult to heal because of the physical condition of the patient, continued external irritation, or the nature of the wound. Promoting healing of common wounds and those presenting difficult circumstances has been important for human development and survival. Throughout history several techniques for healing wounds have been used including, but not limited to: cloth covering or wrapping, plastic strips, pressure devices, some that make use of electrical currents, etc.

The invention may be employed as a healing aid in the treatment of primary or secondary lesions in classifications including, but not limited to: macule, papule, nodule, wheal, plaque, bulla, vesicle, crust, scale, fissure, erosion, ulcer, keloid, scarring, etc.

Adhesive bandages are easy to use, relatively inexpensive and available in a variety of types, shapes and sizes. Adhesive bandages are widely used in hospitals, workplaces and homes to cover or seal and to protect minor wounds from contamination and further injury. Adhesive bandages usually comprise a backing of plastic or fabric, an absorbent wound contact portion and a pressure sensitive adhesive coating to stick the backing to an area proximal to the wound. The pressure sensitive adhesive enhances the utility by making self-application of the bandage a low-tech operation.

Skin provides a natural barrier to foreign substances including chemicals and microbes. Bypassing these natural defenses is thus incorporated into conventional transdermal therapies—including carrier solvents such as DMSO and transdermal injections through needles or liquid streams. The present invention includes embodiments that may feature one or more mixtures of polar lipid(s) such as lecithin, phosphatidylcholine, etc.; one or more biocompatible organic solvents such as an isopropyl palmitate or isopropyl myristate ester; water or other polar solvent; urea or other osmotically active solute; binders or carriers for active ingredients, for example, lipid chains phospholipids, peptides, proteins, etc.; one or more biocompatible surfactants including, but not limited to: docusate sodium, docusate sodium benzoate, etc.; and/or one or more buffers, such as pH controlling buffers, other ion controlling buffers, etc. Additional components may include biocompatible viscosity, stability or other features common in pharmaceutically preparations including, but not limited to: cholesterol, preservatives such as benzyl alcohol, gelling agent, etc.

In one gel embodiment, formulation of the composition comprising one or more pharmaceutically active agent(s), with the pH in a desired acceptable range, the formulation is allowed to thicken producing a gel suitable for topical or other transdermal administration. The range of formulations may include, for example, at least one non-steroidal anti-inflammatory agent that provides local relief from pain and/or other pharmaceutically-active agent including, but not limited to: vascular modifiers, muscle relaxants, etc. Such gel may optionally incorporate a physical protective feature that may not penetrate the skin, but that e.g., may polymerize or otherwise form a bandage or artificial shin-like barrier controlling e.g., access or foreign irritants to the application area (uv light, chemical irritant, microbes, temperature fluctuations, etc.).

### TECHNICAL FIELD OF INVENTION

This invention is directed to patients having wounds and accelerating the healing process of the wound with a wound care ointment, cream, salve or bandage.

In particular, the present invention comprises substances that accelerate communication between cells to help close and heal wounds. The invention is applicable for use for ordinary household type injuries as well as industrial and/or battlefield events, perhaps from chemical accidents or even several types of chemical weapons. One set of tools that accelerate communication between cells to help close and heal wounds are pathways of the endocannabinoid system.

Communication between cells involves multiple natural tools that can be accentuated to improve speed and strength of healing. Hormonal activities such as the fight or flight responses that increase metabolism, change blood flow, stimulate void responses, provide chemical fuels where advantageous, etc. are a long known tool for distant intercellular communication. The entire nervous system is another anciently recognized tool for both local and distant communication. More recently recognized tools include exosomes and tunneling nanotubes that are capable of delivering packets or streams of materials to from one cell to one or a plurality of cells in the delivering cell's vicinity.

Stress is a strong stimulant for TNT formation. For example, malignant cells under ischemic stress release exosomes that stimulate TNT formation. Inflammation in general promotes TNT initiation and growth. TNTs are important promoters of healing at the margins of wounds. Post ischemic recovery in cardiac and central nervous system tissues involves proliferation of TNTs sharing healthy cell components with nearby damaged cells. While TNTs are integral to repairing, redirecting and rebalancing efforts in macroorganisms they do not act alone. Another messenger system that carries small information bearing or corrective molecules within an active range including the ranges in which TNTs operate has as part of its functions stimulating TNTs. Cell membranes, especially membranes of stressed cells bud off small vesicles approximately 1/100 the size of a red blood cell. Since these exosomes are spawned by a cell's plasma membrane, they comprise molecular constituents of their cell of origin which includes membrane lipids, proteins, and often RNA, including mRNAs and/or miRNAs. The exosomal protein composition is determined by the originating cell and so composition analysis can determine cell and tissue of origin. In addition, most exosomes contain an evolutionarily-conserved common set of protein molecules. The protein content of a single exosome ranges up to about 20,000 molecules and many will generally include all or several of these proteins in addition to their more cell specific cargo: HSPA8, CD9, GAPDH, ACTB, CD63, CD81, ANXA2, ENO1, HSP90AA1, EEF1A1, PKM2, AGO2, YWHAE, SDCBP, PDCD6IP, ALB, YWHAZ, EEF2, ACTG1, LDHA, HSP90AB1, ALDOA, MSN, ANXA5, PGK1 and CFL1.

Electronic analogue or digital sensors may be used in some embodiments which meter medicament delivery. Size is not a major constraint, especially with regard to extracorporeal components. Micro or nano scale devices are preferred for their compactness and are especially preferred for subcutaneous or other non-surface placements. A sensor may report back an indication of temperature, pH, salinity, conductance, impedance and/or other parameter to expedite healing on both a grand and local scale.

Exosomes cooperate with TNTs as mediators of cell-to-cell signaling through the transfer of molecules such as mRNAs, microRNAs, and proteins between cells. Exosomes released by healthy cells especially during and after chemical, physical or radiation damage will generally transport several mRNAs and miRNAs along with cytoplasmic and membrane proteins to damaged cells. Exosomes or exosome delivered components can recruit TNTs to provide additional remedial services, such as healthy mitochondria, to help restore health in the damaged cell(s).

Exosomes are rather simple constructs. Essentially, they are lipid bubbles that may have lipoprotein in the membranes and can carry nucleic acid, proteins, ions and cofactors within the bubble. Partially synthetic exosomes are thus readily obtainable using membranes from selected lysed cells and creating vesicles in media compromising the proteins, RNA, sugars, cofactors, ions, etc. to be delivered to the target cell. The target cell can be refined by choosing the plasma donating cell expressing desired membrane proteins or the proteins can be added during vesiculation. The contents may be selected to contain inhibitory proteins, kinases, mRNAs, miRNAs and/or siRNAs as desired to turn on/off and/or up or down regulated one or more metabolic pathways.

In normal situations exosomal release is induced by stress to cells. Depolarization, increasing calcium, heat, especially heat fluctuations, binding and activation receptors on specialized cells are common stimulants of exosome release. Clathrin adaptor AP3 and the v-SNARE TI-VAMP (tetanus neurotoxin-insensitive vesicle-associated membrane protein or VAMP7) are active in lysosome secretion.

When used in the presence of or in conjunction with TNT therapy, the combination has extensive and broad uses. Exosome production may be stimulated to initiate TNT production. Artificial or partially synthetic exosomes may be used as an initiator to stimulate exosome release from specifically targeted cells. The exosome intervention may be used to salvage stressed cells or cells about to undergo stress. Select cell types may be thus primed for tolerance to a potentially damaging therapeutic dose. Exosomes may be used to stimulate TNT facilitated wound healing. These are just a few of the many ways TNTs in conjunction with exosomes have special benefits.

On the other side, exosomes might be used to shut down TNTs when TNTs are at elevated risk of damaging the macroorganism. For example, during or following chemotherapy or after exposure to virus, exosomes can be engineered to deliver one or more inhibitors of TNT formation and/or inhibitory RNAs to limit the TNTs contributions to e.g., restoring vitality to a cell damaged by chemotherapy or closing out viruses from intercellular passaging to expand the viral infection.

A major function of TNTs is to provide connections forming a network of multiple cells such that when one cell is stimulated and its cytoplasmic Ca⁺⁺ increases, this Ca⁺⁺ activation is rapidly spread throughout the network in a process somewhat akin to a neural network but without neurotransmitter involved for the cell to cell activation. Heat, pH, hypoxia, and/or chemical and/or biochemical signaling agents may be advantageously applied in isolation or combination to expedite exchange between cells. Intercellular feedback may cause TNT switching events follow a harmonic cycle.

Lateral gene transfer (LGT) between cells induces exogenous gene expression and may mediate RNA silencing. [Mouse exosomes are internalized and processed by human mast cells (MCs) to express mouse RNA.] The opposite occurs using human exosomes and mouse MCs. Analysis of human MC exosomes found ˜1300 mRNAs and 121 microRNAs (miRNAs) but no DNA or RNA.

Artificial (partially synthetic) exosomes are not limited to natural compounds. The membrane can be engineered to carry selected ligands to precisely interact with only select cells that bind that ligand. The ligand can be a peptide or modified peptide; the ligand may be a small molecule adapted for display on the exosome membrane. The contents can be inhibitory of select processes, toxic to one or more processes, toxic to the cell and/or excitatory to one or more processes. We therefore can use exosomes to deliver most anticipated smaller molecules or complexes to selected cells.

When used in the presence of or in conjunction with TNT therapy, the combination has extensive and broad uses. Exosome production may be stimulated to initiate TNT production. Artificial or partially synthetic exosomes may be used as an initiator to stimulate exosome release from specifically targeted cells. The exosome intervention may be used to salvage stressed cells or cells about to undergo stress. Select cell types may be thus primed for tolerance to a potentially damaging therapeutic dose. Exosomes may be used to stimulate TNT facilitated wound healing. These are just a few of the many ways TNTs in conjunction with exosomes have special benefits.

On the other side, exosomes might be used to shut down TNTs when TNTs are at elevated risk of damaging the macroorganism. For example, during or following chemotherapy or after exposure to virus, exosomes can be engineered to deliver one or more inhibitors of TNT formation and/or inhibitory RNAs to limit the TNTs contributions to e.g., restoring vitality to a cell damaged by chemotherapy or closing out viruses from intercellular passaging to expand the viral infection.

### CANNABINOID SYSTEM

Cannabinoids are short lived lipid compounds produced in various forms in plants and animals. Phyto-cannabinoids (cannabinoids derived from plants) are well known and are the source of the cannabinoid term stemming from THC, the major psychoactive ingredient of marijuana, Cannabis sativa.

Mammals and other animals produce their own versions of cannabinoid substances—substances that react with the receptors that bind THC and related compounds. The cannabinoids produced by an organism to act through these cannabinoid receptors are called endocannabinoids.

Endocannabinoids are active thought our bodies, in the nervous system, the immune system the gastro-intestinal tract, pulmonary system, angiogenesis and virtually every other system.

Cannabinoids are particularly attractive for their ability in stimulating and maintaining tunneling nanotubes (TNTs), as a class of specialized structures (tunnels) connecting individual cells to another cell or connecting multiple cells to form a network of connected cells.

TNTs are extensions of plasma membrane with membrane proteins exposed on the outer and inner surfaces of the tunnel that allows communication of cytoplasm and its contents from one cell to another. Ions and small molecules pass easily following concentration gradients. But these TNTs can also transport relatively huge cellular components, even components as large as mitochondria.

By providing a direct continuous cytoplasmic path between cells, TNTs are adept at communicating and sharing information and activity, especially chemical information and means, such as enzymes and organelles, to use the chemicals.

The direct connection that TNTs provide between cells allows electrical propagation directly from one cell to another absent a synapse as used for cell-to-cell information transfer in the neural system. This direct electrical connection aids in connecting cells at the leading edge of a healing wound; and also can be used to repair metabolically compromised cells surrounding or surrounded by healthy cells. Healthy cells may be cells native to the organism and originally at that location. They may be cells native to the organism but driven using one or more chemotactic factor to the region to be healed. They may be cells native to the organism, but removed and cultured in growth or restorative media before return to the organism to aid healing. Or the cells may be immunologically compatible cells cultured from another source and provided as an aid to healing. The healing cells with their direct electrical connections may exert their effects by activating enzymes, such as voltage-sensitive phosphatase, PI3K and protein kinase A.

The “cannabinoid” (a term indicating cannabis-like activity) compounds have diverse effects, including most notably, some psychoactive effects became known as phytocannabinoids based on their relation to compounds found in the cannabis genus. The endo/phytocannabinoids include but are not limited to: N-acylethanolami(n/d)es which include N-arachidonoylethanolamide (better known as anandamide or more simply AEA), N-palmitoylethanol-amine (PEA), N-linoleoylethanolamide (LEA) and N-oleoylethanolamine (OEA). Since living organisms share many common metabolic paths and features, many of our human endocannabinoids can be found in other species, including plant species. For example, OEA and LEA are in cocoa. Black truffles when grown under certain circumstances contain high levels of AEA. Endocannabinoids are natural to the organism; exocannabinoids are cannabinoids from an external source. Endo- and/or exocannabinoids have different physiologic effects dependent on the endocannabinoid receptor(s) that may be agonized or antagonized.

Phytochemicals (substances found in plants or derivatives of the plant chemicals) or the plants themselves have been recognized to possess biological activities in traditional medical practices. Several classes of compounds with similarities in structure and/or activities to the THC purported active ingredient of the marijuana source plant have been identified. These are available in several plants outside the Cannabis genus and can be, cultured (e.g., through selective breeding or genetic engineering), extracted, purified or synthesized chemically de novo or from derivatives. Such compounds include, but are not limited to:
Cannabigerol class: cannabigerolic acid (CBGA) (antibiotic); cannabigerolic acid monomethylether (CBGAM); cannabigerol (CBG) (antibiotic, antifungal, anti-inflammatory, analgesic); Cannabigerol monomethylether (CBGM); cannabigerovarinic acid (CBGVA); Cannabigerovarin (CBGV).
Cannabichromene class: Cannabichromenic acid (CBCA); Cannabichromene (CBC) (antibiotic, antifungal, anti-inflammatory, analgesic); Cannabichromevarinic acid (CBCVA); Cannabichromevarin (CBCV); Cannabidiolic acid (CBDA) (antibiotic); Cannabidiol (CBD) ((antioxidant, anxiolytic, antispasmodic, anti-inflammatory, analgesic); cannabidiol monomethylether (CBDM); cannabidiol C₄ (CBD-C4); cannabidivarinic acid (CBDVA); cannabidivarin (CBDV); cannabidiorcol (CBD-C1); Δ⁹-tetrahydrocannabinolic acid A (THCA-A); Δ⁹-tetrahydrocannabinolic acid B (THCA-B); 6a,10a-trans-6a,7,8,10a-tetrahydro-6,6,9-trimethyl-3-pentyl-6H-dibenzo[b,d]pyran-1-ol, (Δ⁹ tetrahydrocannabino-, THC) (analgesic, antioxidant, antiemetic, anti-inflammation); Δ⁹-tetrahydrocannabinolic acid-C4 (THCA-C4); Δ⁹-tetrahydrocannabinol-C4 (THC-C4); Δ⁹-tetrahydrocannabivarinic acid (THCVA); Δ⁹-tetra-hydrocannabivarinic (THCV); Δ⁷-cis-isotetrahydrocannabivarin; Δ⁹-tetrahydrocannabiorcolic acid (THCA-C1); tetrahydrocannabiorcol (THC-C1).
Δ⁸-tetrahydrocannabinol class: Δ⁸-tetrahydrocannabinolic acid (Δ⁸-TCA); Δ⁸-tetra-hydrocannabinol (Δ⁸-THC).
Cannabicyclol class: cannabicyclol (CBL); cannabicyclolicacid (CBLA); cannabicyclovarin (CBLV).
Cannabieson class: cannabiesoic acid A (CBEA-A); cannabiesoic acid B (CBEA-B); cannabieson (CBE).
Cannabinol and cannabinodiol class: cannabinolic acid (CBNA); cannabinol (CBN); cannabinol methylether (CBNM); cannabinol-C4 (CBN-C4); cannabivarin (CBV); cannabinol-C2 (CBN-C2); cannabiorcol (CBN-C1); cannabinodiol (CBND); cannabinidivarin (CBDV).
Cannabitriol class: cannabitriol (CBT); 10-Ethoxy-9-hydroxy-Δ-6a-tetrahydrocannabinol (10-EHDT); 8,9-dihydroxy-delta-6a-tetrahydrocannabinol (8,9-DHDT); cannabitriolvarin (CBTV); ethoxy-cannabitriolvarin (CBTVE).
Miscellaneous class: dehydrocannabifuran (DCBF); cannabifuran (CBF); cannabichromanon (CBCN); cannabicitran (CBT); 10-oxo-Δ-6a-tetrahydrocannabinol (OTHC); Δ⁹-cis-tetrahydrocannabinol (cis-THC); 3,4,5,6-tetrahydro-7-hydroxy-α-α-2-trimethyl-9-n-propyl-2,6-methano-2H-1-benzoxocin-5-methanol (2H-iso-HHCV); cannabiripsol (CBR); Trihydroxy-Δ⁹-tetrahydrocannabinol (triOH-THC).
LEA, PEA and OEA will bind to one or more of the endogenous cannabinoid receptors, but they are also important because they maintain AEA activity through their inhibition of the FAAH enzyme that is responsible for degrading AEA. N-alkylamides exert selective effects on the CB₂, and have been shown to exert anti-inflammatory effects similar to AEA. Echinacea contains multiple N-alkylamides that have mimetic effects.

Phytoalkanes, another class of chemical compounds found in various plants, also have demonstrated cannabinolic modulation traits, e.g., N-alkanes ranging from C₉ to C₃₉, 2-methyl-, 3-methyl-, and some dimethyl alkanes are common in spices such as curcumin. The major alkane present in an essential oil obtained by extraction and steam distillation was the N—C₂₉ alkane nonacosane (55.8 and 10.7%, respectively). Other abundant alkanes were heptacosane, 2,6-dimethyltetradecane, pentacosane, hexacosane, and hentriacontane. Curcumin reduces liver fibrosis by modulating cannabinoid receptor transmission.

β-caryophyllene, a phytocannabinoid, and/or its oxides act as full agonists of the CB₂-receptor where they exert anti-inflammatory and analgesic effects that are mediated through CB₂, but not CB₁. Another phytocannabinoid, salvinorin A, from the plant species Salvia divinorum extract is a terpenoid that interacts with a cannabinoid receptor, not yet characterized that apparently forms only in inflammatory conditions. This uncharacterized receptor also acts as a κ-opioid receptor. Many sages produce similar compounds with some activity, but whose activities have not been followed in detail to identify receptor interactions. Myrcene is a major constituent of the essential oil of hops and appears to be related to opioid “high” possibly by agonizing opioid receptors or possibly by antagonizing opioid degradation. Plant sources are hops, verbana and cannabis. Myrcene is also found in lemongrass, thyme and mango. Echinacea contains multiple N-alkylamides that have cannabinoid mimetic effects.

The Helichrysum umbraculigerum, aka woolly umbrella Helichrysum or kerriekruie in Afrikaans, is a fast growing perennial herb with a strong mood-stabilizing and anti-depressant effect due to high concentrations of cannabigerol (CBG). Liverwort contains large amounts of perrottetinenic acid, a THC, mimetic that binds CB₁. The cacao plant has endocannabinoid activity by deactivating the FAAH enzyme thereby maintaining AEA levels and levels of similarly active fatty acid derived molecules. FAAH inhibition combines anti-inflammatory effects of several N-acylethanolamines while it targets additional receptors such as TRPV₁ and peroxisome proliferator activated receptors. TRPV₁ agonists often lend a warmth or physiologic heating sensation and therefore may be used especially when this dual effect is desired.

URB597 is a potent and selective FAAH inhibitor. Inhibiting the FAAH enzyme, a principle degradative enzyme and one involved in synthetic pathways for inflammatory prostaglandins, maintains beneficial cannabinoid levels while reducing adverse effects from breakdown products.

The following list of compounds have been suggested to have cannabinolic activity (i.e., to bind and modulate activity of at least one human cannabinoid receptor): URB597, URB937, AM374, ARN2508, BIA 10-2474, BMS-469908, CAY-10402, JNJ-245, JNJ-1661010, JNJ-28833155, JNJ-40413269, JNJ-42119779, JNJ-42165279, LY-2183240, Cannabidiol, MK-3168, MK-4409, MM-433593, OL-92, OL-135, PF-622, PF-750, PF-3845, PF-04457845, PF-04862853, RN-450, SA-47, SA-73, SSR-411298, ST-4068, TK-25, URB524, URB597 (KDS-4103), URB694, URB937, VER-156084, V-158866, AM3506, AM6701, CAY10435, CAY10499, IDFP, JJKK-048, JNJ-40355003, JNJ-5003, JW618, JW651, JZL184, JZL195, JZP-372A,KML29, MAFP, MJN110,ML30, N-arachi-donoyl maleimide, OL-135, OL92, PF-04457845, SA-57, ST4070, URB880, URB937, indome-thacin, MK-886, resveratrol, cis-resveratrol, aspirin, COX-1 inhibitor II, loganin, tenidap, SC560, FR 122047 hydrochloride, valeryl salicylate, FR122047 hydrate, ibuprofen, TFAP, 6-methoxy-2-naphthylacetic acid, meloxicam, APHS, etodolac, meloxicam, meloxicam sodium salt, N-(4-acetamidophenyl)indomethacin amide, N-(2-phenylethyl)indomethacin amide, N-(3-pyridyl)-indomethacin amide, indomethacin heptylester, SC236, sulinac, sulindac sulfide, pravadoline, naproxen, naproxen sodium salt, meclofenamate sodium, ibupropfen, S-ibuprofen, piroxicam, ketoprofen, S-ketoprofen, R-ibuprofen, ebselen, ETYA, diclofenac, diclofenac diethylamine, flurbiprofen, fexofenadine, pterostilbene, pterocarpus marsupium, 9,12-octadecadiynoic acid, ketorolac (tromethamine salt), NO-indomethacin, S-flurbiprofen, sedanolide, green tea extract (e.g., epicatechin), licofelone, lornoxicam, racibuprofen-d3, ampiroxicam, zaltoprofen, 7-(trifluoromethyl)1H-indole-2,3-dione, aceclofenac, acetylsalicylic acid-d4, S-ibuprofen lysinate, loxoprofen, CAY10589, ZU-6, isoicam, dipyrone, YS121 and MEG (mercaptoethylguanidine) and thus may be appropriate for use in the present invention.

While synthetic cannabinoids should be used with care in the frequency and volume of their dosing, one characteristic of the endo-cannabinolic systems is that they are fantastic self-regulators. For example, exogenous AEA and similar phytocompounds that bind endogenous receptors set in motion pathways to rebalance and restore cannabinoid metabolisms including related pathways for inducing receptors synthetic enzymes and even the degradative enzymes. Small frequent doses can be all the organism requires for superbly balanced cannabinolic controls.

Native, phytomimetic, and/or synthetic cannabinoids can be directly administered to the recipient that may benefit from cannabinolic rebalancing by any suitable means. For example, they may be delivered in a gel, spray, paste, drop, lozenge, a skin patch, eye drops, cream, ointment, etc. There is no restriction on suitable packaging. Another option involves pro-cannabinolic compounds, compounds metabolized by the organism to become cannabinoids which are also suitable as compositions for administering or delivering the active substance.

The endocannabinoid system (ECS), is an important lipid based signaling and immunomodulator system. Lipophilic compounds, those generally non-polar constructs that can readily cross plasma membranes, are prime activators of these endocannabinoid pathways. Research relating to medical uses of marijuana and traditional medicines has shown that at least compounds that bind CB₁ and CB₂ participate in modulating many physiological responses including, but not limited to: appetite, respiration, metabolism, inflammation, allergy, pain, neurotransmission, etc. The ECS is comprised of G-protein coupled receptors (GPCRs) including, but not limited to: CB₁, CB₂,TRPV₁, TRPV₂, TRPV₃, TRPV₄, TRPA₁, TRPM₈, GPR₅₅, GPR₁₁₈, etc.

The native cannabinoid receptor ligands aka “endocannabinoids” are classically represented by arachidonylethanolamide (anandamide, AEA) and 2-arachidonoylglycerol (2AG). Tissue levels of endocannabinoids are maintained by the balance between biosynthesis (e.g., phospholipase D and diacylglycerol lipase-dependent and other pathways), cellular uptake and degradation by enzymes principally, but not limited to: fatty acid amide hydrolase (FAAH) and/or monoacylglycerol lipases (MAGL). Since the discovery of CB₁ and CB₂ GPCRs such as GPR₁₈, GPR₅₅, GPR₁₁₉ and the TRPs have been recognized as members of the cannabinoid family.

These compounds and the proteins responsive to them have important roles in maintaining homeostasis, especially relating to response to smells/odors, food intake, appetite, and external or internal immunologic or allergic response. The endocannabinoids were recognized as the native biomolecules that employ receptors discovered when investigating biologic responses to compounds originating in plants. Originally two cannabinoid receptors were recognized in humans/mammals because THC, a psychoactive cannabinoid substance from Cannabis was found to interact with these proteins. These were dubbed: cannabinoid receptor 1 (CB₁) and cannabinoid receptor 2 (CB₂). AEA and 2AG were recognized as predominant endocannabinoids binding these receptors. CB₁ immunoreactive neurons were found in close proximity to ileal Peyer's patches and were localized in some submucosal blood vessels. However, subsequent discoveries have revealed other endobiologic compounds also binding these receptors and the additional receptors which interact with AEA and 2AG and the additional recognized compounds with endocannabinoid activity.

Activation of CB₂ is generally anti-inflammatory, for example, involved in reduction of NF-κB, AP-1 and inflammatory mediators. CB₂ is primarily expressed on subsets on immune cells and several leukocyte lines of the hematopoietic subsystem (macrophages, both B and T lymphocytes), secondary lymphoid tissues such as spleen, tonsils, Peyer's patches, Lymphatic ganglia, microglia and hepatic myofibroblastic cells.

Endocannabinoids, in general, often through CB1 and/or CB2, inhibit inflammatory responses of resident and infiltrating immune cells. Other G-protein coupled receptors active in the endocannabinoid systems include, but are not limited to: TRPV₁, TRPV₂, TRPV₃, TRPV₄, TRPA₁, TRPM₈, GPR₅₅, GPR₁₁₈, etc.

In addition to assistance in healing through nanotubule formation and maintenance, the anti-inflammatory activity enables the healing process to continue without immune interference. CB1/CB2 knock-out mice suffer from exacerbated allergic responses. In addition to assistance in wound healing, materials of the present invention are also appropriate for helping recover from allergic or other immune stimulated episodes.

As another benefit, the analgesic effect of cannabinoids when applied to the wounded, damaged or inflamed tissues can further the healing process by reducing mechanical damage from scratching or similar activities.

Two rather specific cannabinoid receptors, CB₁ and CB₂, have been identified and are targeted by numerous exogenous and endogenous cannabinoid ligands. Activation of mast cell CB₂ has direct anti-inflammatory effects, causing decreased release of pro-inflammatory mediators by these cells. Activation of CB₁ on bronchial nerve endings has bronchodilator effects acting on the airway smooth muscle with benefits for treating airway hyperreactivity and asthma. Pharmacologic interference using endocannabinoid inhibitors reduces pain and inflammation. This is mediated at least by CB₁ and CB₂. Activation of CB₁ in cerebral blood vessels has beneficial anti-inflammatory/anti-ischemic effects.

GPR₅₅ and CB₁ receptors modulate each other's signaling properties. GPR₅₅ forms heteromers with another 7× transmembrane spanning/GPCR which then interacts with CB₁. GPR₅₅-CB₁ heterodimer acts as a modified cannabinoid receptor that cells form to modulate activities in response to exogenous cannabinoid. This plasma membrane response is independent of cannabinoid effects on internal organelles including, but not limited to: mitochondria, peroxisomes, endoplasmic reticulum, golgi, etc.

CB₁ and CB₂ are both expressed on Mast Cells (MC) and CB₂ is on Eosinophil (Eo) membranes. CB₁ and CB₂ have demonstrated anti-inflammatory effects on MCs. CB₁ downregulates MC degranulation, and CB₂ downregulates pro-inflammatory mediator release. Antagonizing CB₁ on the MCs stimulates degranulation and increases cell numbers without affecting MC proliferation. CB₁ activation of bronchial nerve endings has bronchodilatory effects and therefore proves to be beneficial in asthmatic response therapy. 2AG and the synthetic selective agonist JWH-133 induce Eo chemotaxis, shape change, adhesion production of reactive oxygen species and increase in CD11b expression, via CB₂ activation.

Although the ECS has multiple involvements, the endocannabinoids, in general, are produced near where they are needed. With respect to skin and healing or repair of wounds or of allergic or autoimmune presentations, cannabinoids appear to be beneficial. For example, CB₂ is expressed in a time dependent manner post injury and during skin wound healing in mice.

While not intending the invention to be bound by this hypothetical mechanism, TNT-dependent transference of calcium signals appears important in stimulating nascent cell production, growth and healing. TNT membrane passages may also provide nutrients and enzymes to the developing cells simplifying their demands and coordination intricacies for massive amounts of transcription, translation and post translation processing. Thus, TNTs are important for their activities as facilitators of healing processes. The present invention features accelerating healing by aiding development of the TNTs and associated bio events involved in healing processes.

For instance, based on research reported by Chifflet et al, 2005; Wood et al, 2002; and Zhao et al, 2006, TNT-mediated electrical coupling might be involved in the wound-healing process. According to this research, the healing mechanism involves cytoplasmic extensions that are enriched in F-actin and connect opposite cells, as well as the occurrence of membrane depolarization at the leading edge of the wound.

The technology required for practicing the present invention is known in the art and easily adaptable to present purposes. For example, as provided in the discussion above, cannabinoids and cannabinoid derivatives are well-known in the art along with means for purifying and producing desired cannabinoid active agents. Yeast or other in vitro systems are available and/or can be engineered using conventional technologies to synthesize phyto- and animal-derived cannabinoid compounds.

Delivery to the wound area is also easily accomplished with conventional techniques. A preferred embodiment, preferred for its simplicity, is applying the cannabinoid containing treatment composition or supplements as a spray. The spray method can be by any conventional means including, but not limited to: a pump spray, pressurized spray, etc., generally with a carrier liquid which may coexist with a drying agent. A preferred spray also encloses or covers the wound; more preferably, a spray comprises a resorbable material. Some versions of the spray product may remain intact after washing the site. Some versions may wash off with water, soap and water, alcohol or the like. Sprays may be formulated to be applied as a long lasting—several day—application, or may be formulated for repeated or multiple applications. The invention is not constrained to a particular schedule, but may be designed for one-time application, daily application, 2, 3, 4, 5, 6, 8, 12, times daily, even hourly or more frequent application if desired for appearance, comfort or whatever.

Some embodiments may feature specialized transdermally effective components, for example comprising a formulation including one or more polar lipid(s) (such as lecithin, phosphatidylcholine, etc.), a biologically acceptable organic solvent (such as isopropyl palmitate, isopropyl myristate esters, sucrose esters, etc.), a surfactant (such as docusate sodium, docusate sodium benzoate, docusate calcium, tween 80, polysorbate 80, etc.), water, and urea, at a pH of between about 6.0 and 8.0 and preferably between 6.0 and 7.0. In addition, the composition may optionally include cholesterol or a preservative such as benzyl alcohol.

Conventional delivery facilitators including, but not limited to: Lipoderm®, an HRT Cream Base, Versabase®, hydroalcohol gel, etc., may be used in one or more embodiments of the present invention.

When used in pharmaceutical or substance delivery preparations, preferably, the lecithin, phosphatidylcholine, etc. is of a high quality, market accepted, pharmaceutical grade. Appropriate lecithin and phosphatidylcholine maybe obtained as commercially available soya lecithin or soya phosphatidylcholine. For some embodiments, preferably, soya lecithin is used in the composition of this invention.

A biologically or pharmacologically acceptable organic solvent used in compositions of the present invention may comprise any non-toxic solvent wherein other components, e.g., when present, the polar lipid, the pharmaceutically active compound and urea are soluble. Preferably said solvent assists as a solubilizing vehicle for carrying pharmaceutically active compounds across the skin preferably mammalian skin. Acceptable esters for this purpose include, but are not limited to isopropyl esters, e.g., isopropyl myristate, isopropyl palmitate, etc.

One example for preparing a composition of the present invention may incorporate a method mimicking or similar to one wherein a polar lipid is dissolved in organic solvent at mass ratios anywhere from about 5:1 to 1:5. Polar lipid and organic solvent may be mixed in even mass ratios. As an example, in one embodiment of the invention, soya lecithin and isopropyl myristate may be combined in equal mass ratios and mixed until the lecithin is evenly distributed in isopropyl myristate. Such mixture is quite stable and therefore may be archived and used after prolonged storage without loss of activity.

Continuing, after the solvent-polar lipid mixture is thoroughly dispersed, one or more pharmaceutically active compound(s) may be added and dissolved. Heating an aliquot of the solvent-polar lipid mixture may facilitate dissolution. Then adding, e.g., on a mass basis, an amount of active compound equal to about 0.01 to 30% of the mass of the solvent-polar lipid and mixing for sufficient interval results in proper dissolution. In addition to cannabinolically active substances such composition may optionally include one or more topical anaesthetics such as lidocaine, a steroidal anti-inflammatory such as cortisone. a peptide, protein, hormone such as platelet factor 4, a substance P antagonist such as capsaicin, a muscle relaxant such as cyclobenzaprine, an antifungal compound such as nifedipine, Fluconazole®, etc., an anti-inflammatory analgesic such as diclofenac sodium.

In some preferred embodiments, a surfactant for example, at a concentration of between about 1-20% of the final composition mass may promote dissolution, stability, etc. In several embodiments, the composition may include non-skin penetrating substances that while remaining outside the skin may polymerize or otherwise interact to form a protective barrier over the delivery site.

The resultant treatment composition may include or result in a deformable protectant material that is characterized by a Young's modulus in a range between about 0.1 kPa and 1000 kPa, or more refined in ranges of about 0.5 kPa and 500 kPa, about 1 kPa and 250 kPa, about 2 kPa and 100 kPa, about 5 kPa and 100 kPa, about 10 kPa and 50 kPa or about 25 kPa and 40 kPa.

Some embodiments may comprise urea, preferably as an aqueous solution, admixed with the solvent-polar lipid mixture. The urea is added for example in a concentration and volume so that the resultant urea concentration will be between about 5% and 20% by mass of the final composition mass. Thus, using a 20% aqueous solution of urea, about 10 grams is added to about 100 grams of the solvent-polar lipid mixture with dissolved pharmaceutically active compound. In some instances, the pharmaceutically active agent will more readily dissolve if added after addition of the urea, and in other instances before the addition of urea. In any event, The amount and method of adding urea is a choice routinely made one skilled in the art taking into account the particular formulation being prepared and the solubility characteristics of the particular pharmaceutically active compound being solubilized.

Upon formulation of the above described composition with the pharmaceutically active agent, the pH is adjusted to a biologically acceptable pH, generally about a pH of 5.8 to 7.4, and often preferably about 6.0 to 7.0. Addition of buffering peptides or proteins, acetic or citric acid or other biological buffer such as sodium carbonate or triethanolamine may facilitate pH management. Once the composition reaches acceptable predetermined pH in preferred embodiments, the formulation thickens to form a gel for topical administration.

In one embodiment of the invention, a composition is formulated with a non-steroidal anti-inflammatory agent, such as ibuprofen or ketoprofen to modulate cannabinoid activities. Such formulation when transdermally prepared as described herein is rapidly absorbed through the skin and thereby may provide local relief from pain as healing progresses.

For ease of preparation, it may be preferred to prepare a first gel composition, hereinafter “base gel”, as a stock to be used for addition of other components in preparing a final composition for topical administration. There are several possible formulations of the base gel. For example, a base gel may be prepared by mixing lecithin organogel (L.O.), as a 1:1 (m/m) mixture of lecithin and isopropyl myristate, with LID oil (a 1:1 [m/m] mixture of L.O. and docusate sodium), dissolving additional docusate sodium powder into this mixture, and then adding aqueous urea.

In one embodiment of the base gel formulation, the final concentrations may be about: L.O.=25%; docusate sodium=15%; urea=10%; and water=50%. These ratios may be varied e.g., such that the final amounts of each component are as follows: L.O.=20-30%; docusate sodium or another surfactant=10-20%; urea=5-20%; and water=30-60%. The base gel may then be added to solubilized active ingredients and other excipients which may be useful in solubilizing the active ingredient, such as DMSO, peppermint oil, glycerin, and/or polyethylene glycol. A homogenous mixture may then be prepared by carefully blending the various components.

After the formulations such as those described herein as examples have been prepared, beneficial use of the formulations is simply a matter of applying the formulation to affected areas where transdermal delivery of the incorporated pharmaceutically active agent is desired. T

[symbol]

A compositional spray may be applied by any means including, but not limited to: spritzer, compressed propellant, additive manufacturing device, aerosol, pump spray, etc.

A spray may comprise, in addition to a compound that stimulates cannabinoid receptor activity, an antiseptic, an antibiotic, a coloring agent, an anesthetic, an analgesic, a drying agent, a coolant, an ultraviolet light absorbent and/or other emollient suitable for skin. Skin sprays are known in the art, for example, Elastoplast® spray plaster, Nobecutame® (trademark registration expired in US 1996), Germolene®, Sprüh-pflaster®, Elastoplast®, Nexcare™, etc. Coolants may include evaporative coolants and/or compounds that give a cooling sensation including, but not limited to: menthol, 2-isopropyl-N,2,3-trimethylbutyramide,N-ethyl-p-menthane-3-carboxamide, ethyl 3-(p-menthane-3-carboxamido)acetate, 1R,25,5R)-N-(4-methoxyphenyl)-p-menthanecarboxamide, N-ethyl-2,2-diisopropylbutanamide, N-cyclopropyl-5-methyl-2-isopropylcyclohexanecarboxamide, N-(1,1-dimethyl-2-hydroxyethyl)-2,2-diethyl-butanamide, menthoxyethanol, N-(4-cyanomethylphenyl)-p-menthanecarboxamide, N-(2-(pyridin-2-yl)ethyl)-3-p-menthanecarboxamide, N-(2-hydroxyethyl)-2-isopropyl-2,3-dimethyl-butanamide, N-(4-(carbamoylmethyl)phenyI)-menthylcarboxamide, 2S,5R)-N-[4-(2-Amino-2-oxoethyl)phenyl]-p-menthanecarboxamide, 1R,2S,5R)-N-(4-methoxyphenyl)-p-menthanecarboxamide, N-cyclopropyl-5-methyl-2-isopropylcyclohexanecarbonecarboxamide, 2-[(2-p-menthoxy)ethoxy]ethanol, 2,6-diethyl-5-isopropyl-2-methyltetrahydropyran, trans-4-tert-butyl-cyclohexanol, 5-methyl-2-(propane-2-yl)cyclohexyl-N-ethyloxamate, N-ethyl-p-menthane carboxamide, N-2,3-trimethyl-2-isopropyl butane amide, menthyl lactate, menthone glycerine acetal, mono-menthyl succinate, mono-menthyl glutarate, 0-menthyl-glycerine, menthyl-N,N-dimethyl succinamate, N-(4-cyano methyl phenyl)-p-menthane carboxamide, N-(2-(pyridin-2-yl)ethyl)-3-p-menthane carboxamide, (I-menthoxy)-1,2-propanediol, etc.

The spray may comprise water and/or other carrier substance or solvent, with, in addition to the active cannabinoid(s), components such as polyvinylpyrrolidone, dimethylether, acrylic copolymer, polyurethane polymer, cellulose nitrate, benzocaine, hexamethyldisiloxane, isooctane, acrylate terpolymer, polyphenylmethylsiloxane, benzethonium chloride, sodium benzoate, acetone, amylacetate, tetramethylthiuram disulphide, castor oil, drometrizole, ethylacetate, 8-hydroxyquinoline, nitrocellulose, sd alcohol 40, dyclonine hydrochloride, oil of cloves, pyroxylin solution, bacitracin, erythromycin, silver sulfadiazine, retapamulin, mupiocin, beomycin, polymyxin(b), polysporin, mafenide, aminoglycosides (including, but not limited to: amikacin, gebtamicin, kanamycin, neomycin, netimicin, tobramycin, paromycin, streptomycin, spectinomycin, etc.), perfumes, colorant, odor maskers, etc.

A carrier may evaporate and allow the discharge to gel or solidify. The carrier may react or contribute to one or more reactions that form a protective coat or barrier. Multiple applications are within the scope of the invention. Multiple applications may be identical or may be differentiated by layering or timing. For example, a first “blue” spray may be applied initially and include a physiologic coolant sensation agent, an anesthetic or the like, while a second “yellow” may lack this or these but include additional cannabinoid and perhaps antiseptic, a third “red” may include, as a cannabinoid, a cannabinoid active on the TRPV1 receptor and thereby provide a calming warmth sensation, a fourth “green” may serve chiefly as a shield against water or other external challenge, a fifth “violet” may serve a camouflage or decorative function, etc.

Multiple applications are within the scope of the invention. Multiple applications may be identical or may be differentiated by layering or timing. For example, a first “blue” spray may be applied initially and include a physiologic coolant sensation agent, an anesthetic or the like, while a second “yellow” may lack this or these but include additional cannabinoid and perhaps antiseptic, a third “red” may include, as a cannabinoid, a cannabinoid active on the TRPV1 receptor and thereby provide a calming warmth sensation, a fourth “green” may serve chiefly as a shield against water or other external challenge, a fifth “violet” may serve a camouflage or decorative function, etc.

While several aspects that may be used in or with this invention are considered in the text of this application, these aspects are not to be considered exclusive. The various aspects may be applied in a conjoint application, may be applied in parallel, e.g., from a plurality of source applicators together in time, and/or may be applied sequentially.

Accordingly, one aspect of this invention provides a treatment composition that accelerates healing of a surface lesion. Such treatment composition may comprise a topical formulation with at least one active ingredient that binds to and activates at least one endogenous cannabinoid receptor.

The invention may also incorporate a protectant material that solidifies, gels or otherwise remains over the wound surface. This covering may protect the recovering or healing skin or other integument from additional trauma such as UV damage, mechanical damage, infection, irritation, etc. The covering perhaps may hide or disguise the injury, affliction, lesion, etc. and thereby avoid notice.

The protectant material may be resorbable, for example, comprise a resorbable substance which may have resorption time controllably selected, perhaps to avoid a requirement for further attention or to make room for additional applications without undue thickness.

Aspects of this invention may provide a protectant material that remains after a rinsing or washing. The coating may form a washable substance allowing hygienic treatments without necessity for reapplication.

Several aspects of this invention include a formulation that is applicable in a spray dispenser.

These and other aspects of the invention include a protectant material that comprises or forms a colorant.

Some aspects may include an antiseptic.

These or other aspects may include one or more antibiotic substance.

Some aspects may include an anesthetic.

Some aspects may include an analgesic. A preferred embodiment may include an analgesic cannabinoid compound.

One or more drying agents may be used in or in addition to the cannabinoid application.

One or more coolant agent may be used in or with the applied substance(s).

Some aspects may incorporate an ultraviolet light absorbent material as a protectant form solar or other UV sourced damage.

Various formulations of the present dispensed materials may incorporate or include a skin emollient.

The coatings may be configured to have. for example, a Young's modulus between about 0.1 kPa and 1000 kPa.

Some aspects may present with a Young's modulus between about 0.5 kPa and 500 kPa.

These and other aspects may include protectant material(s) with a Young's modulus between about 1 kPa and 250 kPa.

Refinements may sport a Young's modulus between about 2 kPa and 100 kPa.

For protecting integument, the dispensed material(s) may result in a coating with a Young's modulus between about 5 kPa and 100 kPa.

In some embodiments, the protectant material may have a Young's modulus between about 10 kPa and 50 kPa.

The protectant material may in some cases have a Young's modulus between about 25 kPa and 40 kPa.

Whether with or in a coating or separate from embodiments with a protectant shielding aspects of this invention may include at least one component selected from the group consisting of: an antiseptic, an antibiotic, a coloring agent, an anesthetic, an analgesic, a drying agent, a coolant sensation agent, an ultraviolet light absorbent and skin emollient.

Embodiments of the invention may include one or more molecules selected from the group consisting of mammalian cannabinoids.

Preferred embodiments of the invention might be formulated to result in increased presence or activity of at least one active ingredient comprises at least one molecule selected from the group consisting of: AEA, 2AG, PEA, OEA and LEA.

These and other embodiments may include at least one active ingredient derived from or being at least one molecule selected from the group consisting of: phyto-cannabinoids, biosimilars and synthetic cannabinoids.

One or more embodiments of the present invention may have at least one active ingredient selected from the group consisting of: URB597, URB937, AM374, ARN2508, BIA 10-2474, BMS-469908, CAY-10402, JNJ-245, JNJ-1661010, JNJ-28833155, JNJ-40413269, JNJ-42119779, JNJ-42165279, LY-2183240, cannabidiol, MK-3168, MK-4409, MM-433593, OL-92, OL-135, PF-622, PF-750, PF-3845, PF-04457845, PF-04862853, RN-450, SA-47, SA-73, SSR-411298, ST-4068, TK-25, URB524, URB597 (KDS-4103), URB694, URB937, VER-156084, V-158866, AM3506, AM6701, CAY10435, CAY10499, IDFP, JJKK-048, JNJ-40355003, JNJ-5003, JW618, JW651, JZL184, JZL195, JZP-372A,KML29, MAFP, MJN110,ML30, N-arachidonoyl maleimide, OL-135, OL92, PF-04457845, SA-57, ST4070, URB880, URB937, indomethacin, MK-886, resveratrol, cis-resveratrol, aspirin, COX-1 inhibitor II, loganin, tenidap, SC560, FR 122047 hydrochloride, valeryl salicylate, FR122047 hydrate, ibuprofen, TFAP, 6-methoxy-2-naphthyl acetic acid, meloxicam, APHS, etodolac, meloxicam, meloxicam sodium salt, N-(4-acetamido-phenyl)indomethacin amide, N-(2-phenylethyl)indomethacin amide, N-(3-pyridyl)indomethacin amide, indomethacin heptyl ester, SC236, sulinac, sulindac sulfide, pravadoline, naproxen, naproxen sodium salt, meclofenamate sodium, ibupropfen, S-ibuprofen, piroxicam, ketoprofen, S-ketoprofen, R-ibuprofen, ebselen, ETYA, diclofenac, diclofenac diethylamine, flurbiprofen, fexofenadine, Pterostilbene, Pterocarpus marsupium, 9,12-octadecadiynoic acid, Ketorolac (tromethamine salt), NO-indomethacin, S-flurbiprofen, sedanolide, green tea extract (e.g., epicatechin), licofelone, lornoxicam, rac ibuprofen-d3, ampirxicam, zaltoprofen, 7-(trifluoro-methyl)1H-indole-2,3-dione, aceclofenac, acetylsalicylic acid-d4, S-ibuprofen lysinate, loxo-profen, CAY10589, ZU-6, isoicam, dipyrone, YS121 and MEG (mercaptoethylguanidine). Preferred embodiments may incorporate 2, 3, 4, 5, 6 or even more cannabinolic supportive compounds or enzymes.

Cannabinoids have been studied and compared to results in several general categories of active substances. Thus treatment compositions may include one or more active ingredient that is is a member of a class of cannabinoids selected from the group consisting of: Cannabigerol class, Cannabichromene class, Cannabicyclol class, Δ8-tetrahydrocannabinol class, Cannabieson class, Cannabinol and cannabinodiol class, Cannabitriol class and Miscellaneous class.

Preferred embodiments may comprise at least one molecule selected from the group consisting of: CBGA, CBGAM, CBG, CBGM; CBGVA and CBGV.

These or additional preferred embodiments may comprise at least one molecule selected from the group consisting of: CBCA, CBC, CBCVA, CBCV, CBDA, CBD, CBDM, CBD-C4, CBDVA, CBDV, CBD-C1, THCA-A, THCA-B, 6a,10a-trans-6a,7,8,10a-tetrahydro-6,6,9- trimethyl-3-pentyl-6H-dibenzo[b,d]pyran-1-ol, THC,) THCA-C4, THC-C4, THCVA, THCV, Δ7-cis-isotetrahydrocannabivarin, THCA-C1 and THC-C1.

Aspects may include at least one molecule selected from the group consisting of: Δ8-TCA and Δ8-THC.

Together or separately some aspects of the invention may be formulated to result in at least one active ingredient that comprises at least one molecule selected from the group consisting of: CBL, CBLA and CBLV.

Some aspects may specifically include at least one active ingredient comprising at least one molecule selected from the group consisting of: CBEA-A, CBEA-B and CBE; some aspects may specifically include at least one active ingredient comprising at least one molecule selected from the group consisting of: CBNA, CBN, CBNM, CBN-C4, CBV, CBN-C2, CBN-C1, CBND and CBDV; some aspects may specifically include at least one active ingredient comprising at least one molecule selected from the group consisting of: CBT, 10-EHDT, 8,9-DHDT, CBTV and CBTVE; some aspects may specifically include at least one active ingredient comprising at least one molecule selected from the group consisting of: DCBF, CBF, CBCN, CBT, OTHC, cis-THC, 2H-iso-HHCV, CBR and triOH-THC.

Sourcing of the active ingredient(s) is not a restraint of the operation of this invention. Embodiments may therefore include at least one active ingredient from at least one molecule derived from a source selected from the group consisting of: echinacea, echinacea purpurea, echinacea angustifolia, curcurmin, Salvia divinorum, sage, lemon grass, hops, verbana, cannabis, thyme, mango, helichrysum umbraculigerum, liverwort, cacao, ginger, tumeric, curcuma longa, magnolia officinalis, norway spruce, black pepper, basil, myristica fragrans, cloves, sciadopitys verticillata, oregano, cinnamon, black pepper, hemp, rosemary, flax and elettaria repens.

Molecules contributing to activity of the inventive treatments may be selected from the group consisting of: β-caryophyllene, a β-caryophyllene oxide, salvinorin A, myrcene, perrottetinenic acid, apigenin, quercetin, cannflavin A, cannflavin B, β-sitosterol, vitexin, isovitexin, kaempferol, luteolin, orientin, a gingerol, capsaicin, curcumin, demethoxycurcumin, bisdemethoxycurcumin, cyclocurcumin, trans-resveratrol, diferuloylmethane, trans-arachidins, transpiceatannol, isoprenylated trans-resveratrol derivatives, sciadonic acid magnolol, honokiol, malyngamide B, (+) sabinene, (−) sabinene, isobutylamide, dodeca-2E,4E-dienoic acid isobutylamide, dodeca-2E,4E,8Z,10Z-tetraenoic acid alkylamide, 1-[(2E,4E,8Z)-tetradeca-trienoyl] piperidine, β-caryophyllene and ajulemic acid.

Suitable biologic targets for component(s) of the present invention include at least one endogenous cannabinoid receptor selected from the group consisting of: CB1 and CB2.

One or more target(s) of the present invention may be at least one endogenous cannabinoid receptor that is or can be classified as a G-protein coupled receptor.

Suitable biologic targets for component(s) of the present invention include at least one endogenous cannabinoid receptor selected from the group consisting of: CB₁, CB₂, TRPV₁, TRPV₂, TRPV₃, TRPV₄, TRPA₁, TRPM₈, GPR₁₈, GPR₁₁₉, GPR₅₅ and GPR₁₁₈.

In one or more embodiments of the present invention, the composition may be formulated to comprise an antibacterial agent, for example, bacitracin or another desired antibiotic. Transdermal delivery of such antibacterial agent to sites of healing and possible infection may further promote healing.

Exemplary compositions of the invention may deliver a concentration between about 0.01% to 30% or possibly more by weight of active compound(s). Compositions comprising a plurality of two or more biologically active ingredients may be administered to a recipient desiring or believed to benefit from more than a single active treatment mode at any one or more localized spot(s). Depending on nature of injuries, a recipient may receive different formulations comprising differing active compounds at various wound sites.

One or more compositions of the present invention may be applied topically as frequently as required absent local adverse reactions or toxicity related the active ingredient(s) or delivery vehicle.

A preferred embodiment may include at least one ingredient that binds TRPV₁.

Preferred embodiments may have at least one active ingredient that is obtained from a phyto-cannabinoid.

Preferred embodiments may include at least one active ingredient that is a synthetic cannabinoid.

Preferred aspects include those where the treatment composition is provided in a spray format.

Other preferred aspects include those where the treatment composition is provided in a patch format.

Some preferred aspects may include those where the treatment composition is provided in a cream format; some preferred aspects may include those where the treatment composition is provided in a ointment format; some preferred aspects may include those where the treatment composition is provided in a stick format; some preferred aspects may include those where the; treatment composition is provided in a solid format; some preferred aspects may include those where the treatment composition is provided in a liquid format; and some preferred aspects may include those where the treatment composition is provided in a drop format.

At least one embodiment features a coolant agent selected from the group consisting of: menthol, 2-isopropyl-N,2,3-trimethylbutyramide, N-ethyl-p-menthane-3-carboxamide, ethyl 3-(p-menthane-3-carboxamido)acetate, 1R,2S,5R)-N-(4-methoxyphenyl)-p-menthane-carboxamide, N-ethyl-2,2-diisopropylbutanamide, N-cyclopropyl-5-methyl-2-isopropyl-cyclohexanecarboxamide, N-(1,1-dimethyl-2-hydroxyethyl)-2,2-diethylbutanamide, menthoxyethanol, N-(4-cyanomethylphenyl)-p-menthanecarboxamide, N-(2-(pyridin-2-yl)ethyl)-3-p-menthanecarboxamide, N-(2-hydroxyethyl)-2-isopropyl-2,3-dimethylbutanamide, N-(4-(carbamoylmethyl)-phenyl)-menthylcarboxamide, 2S,5R)-N[4-(2-Amino-2-oxoethyl)phenyl]-p-menthanecarboxamide, 1R,2S,5R)-N-(4-Methoxyphenyl)-p-menthanecarboxamide, N-Cyclopropyl-5-methyl-2-isopropylcyclohexanecarbonecarboxamide, 2-[(2-p-menthoxy)-ethoxy]ethanol, 2,6-Diethyl-5-isopropyl-2-methyltetrahydropyran, trans-4-tert-butylcyclo-hexanol, 5-methyl-2-(propane-2-yl)cyclohexyl-N-ethyloxamate, N-ethyl-p-menthane carboxamide, N-2,3-trimethyl-2-isopropyl butane amide, menthyl lactate, menthone glycerine acetal, mono-menthyl succinate, monomenthyl glutarate, O-menthyl-glycerine, menthyl-N,N-dimethyl succinamate, N-(4-cyano methyl phenyl)-p-menthane carboxamide, N-(2-(pyridin-2-yl)ethyl)-3-p-menthanecarboxamide and (I-menthoxy)-1,2-propanediol.

Aspects of the invention include those applied by spritzer, compressed propellant, additive manufacturing device, aerosol, pump spray, etc.

Preferred methods of the invention include those that accelerate healing of a surface lesion comprising applying a composition comprising at least one active ingredient that binds and activates at least one endogenous cannabinoid receptor to a surface lesion.

Some embodiments may include serial or repeated applications.

The frequency of repetition is not limiting for example, application may be repeated daily.

Other aspects include those where application may be repeated at least at 2 times daily, 3 times daily, 4 times daily, 5 times daily, 6 times daily, 8 times daily and/or 12 times daily.

Rate of application(s) is not limited to daily consideration. For example, applications may be repeated after about an interval selected from the group consisting of: five minutes, ten minutes, fifteen minutes, thirty minutes, an hour, two hours, three hours, four hours, six hours, eight hours, 12 hours, 24 hours, 36 hours, 48 hours, 60 hours, 72 hours and 84 hours, etc.

What is claimed is:

1. A treatment composition to accelerate healing of a surface lesion, said treatment composition comprising a topical formulation with at least one or a plurality of components capable of binding and activating at least one endogenous cannabinoid receptor, said activating stimulating tunneling nanotubule intercellular connectivity.

2. The composition of claim 1 comprising at least one pharmaceutically acceptable organic solvent, at least one polar lipid, at least one surfactant, and at least one compound capable of activating a cannabinoid receptor.

3. The composition of claim 1 further comprising water wherein the pH of said composition is between about 6.0 and about 8.0.

4. The composition of claim 1 wherein said polar lipid is selected from the group consisting of: lecithin and phosphatidylcholine.

5. The composition of claim 1 wherein said pharmaceutically acceptable organic solvent comprises an isopropyl ester.

6. The composition of claim 5 wherein said isopropyl ester is selected from the group consisting of isopropyl myristate and isopropyl palmitate.

7. The composition of claim lfurther comprising urea at a concentration of about 5% to about 20% by mass of the final composition.

8. The composition of claim 4, wherein said polar lipid comprises lecithin.

9. The composition of claim 2, wherein said surfactant is selected from the group consisting of docusate sodium, docusate sodium benzoate, docusate calcium, tween 80 and polysorbate 80.

10. The composition of claim 1, further comprising at least one pharmaceutically active substance selected from the group consisting of: an analgesic substance, an anti-inflammatory substance, a biologically active protein, a substance P antagonist and a muscle relaxant.

11. The composition of claim 10 wherein said pharmaceutically active substance comprises a non-steroidal anti-inflammatory agent.

12. The composition of claim 2 comprising lecithin, isopropyl myristate, urea and a surfactant.

13. The composition of claim 12 comprising lecithin at a concentration of 10-30%, isopropyl myristate at a concentration of 10-30%, urea at a concentration of 5-20%; and a surfactant at a concentration of 10-20% by mass in an aqueous composition.

14. The composition of claim 13 wherein said aqueous composition comprises about 30% to about 60% water.

15. The composition, of claim 10 comprising cyclobenzaprine.

16. The composition of claim 2 comprising about:
10-15% lecithin;
10-15% isopropyl myristate;
10-20% docusate sodium;
5-15% urea;
25- 60% water 25-60%; and
at least one pharmaceutically active ingredient present at between 0.01-30%.

17. A method of making the composition of claim 2, said method comprising:
(a) dissolving a polar lipid in a pharmaceutically acceptable organic solvent to produce an organic base;
(b) adding at least one surfactant to said organic base said adding producing a surfactant concentration of about 10% to about 20%;
(c) dissolving at least one pharmaceutically active compound in the surfactant mixture of (b);
(d) adding water and (e) adjusting pH to between about 6.0 and about 8.0.

18. The method of claim 17 wherein said surfactant is added during or after said (d) adding water.

19. The method of claim 17 further comprising urea to a concentration of about 5% to about 20%.

20. The composition of claim 2 further comprising a protectant material that solidifies, gels or otherwise remains at the wound surface.

21. The composition of claim 20 wherein said protectant material is applicable in a spray formulation.

22. The composition of claim 2 wherein said at least one active ingredient comprises at least one molecule selected from the group consisting of: phyto-cannabinoids, biosimilars and synthetic cannabinoids.
